(19) United States
(12) Patent Application Publication
(10) Pub. No.: US 2007/0292368 A1
(43) Pub. Date: Dec. 20, 2007

(54) ORAL CARE COMPOSITION

(76) Inventors: Tim Clarot, Phoenix, AZ (US); Regina Miskewitz, Phoenix, AZ (US)

Correspondence Address: SNELL & WILMER L.L.P. (Main), 400 EAST VAN BUREN, ONE ARIZONA CENTER, PHOENIX, AZ, 85004-2202, US

(21) Appl. No.: 11/748,653

(22) Filed: May 15, 2007

Related U.S. Application Data
(60) Provisional application No. 60/800,638, filed on May 15, 2006.
(60) Provisional application No. 60/800,639, filed on May 15, 2006.
(60) Provisional application No. 60/800,918, filed on May 15, 2006.
(60) Provisional application No. 60/800,636, filed on May 15, 2006.

(51) Int. Cl. A61K 8/00; A61K 8/18; A61K 8/97; A61P 43/00

(52) U.S. Cl. 424/53; 424/49; 424/58

(57) ABSTRACT

A composition for improving oral health and a method of using and forming the composition are disclosed. The composition includes one or more active ingredients and a carrier configured to maintain the composition in contact with a surface for an extended period of time to promote oral health and hygiene.

### CROSS REFERENCE TO RELATED APPLICATIONS

001 This application claims the benefit of U.S. Patent Application Ser. No. 60/800,638, entitled METHOD AND SYSTEM FOR FACILITATING ORAL HEALTH, filed May 15, 2006; Ser. No. 60/800,639, entitled ORAL CARE PROTECTION SYSTEM, filed May 15, 2006; Ser. No. 60/800,918, entitled COMPOSITION TO PREVENT AND REDUCE TARTAR BUILDUP AND METHOD OF USING SAME, filed May 15, 2006; and Ser. No. 60/800,636, entitled ORAL CARE GEL COMPOSITION FOR IMPROVING OR MAINTAINING ORAL HEALTH, filed May 15, 2006.

### FIELD OF INVENTION

The present invention generally relates to compositions for promoting oral health and hygiene. More particularly, the invention relates to compositions for promoting oral health, such as through the prevention of or reduction of tartar, plaque, gingivitis, and other gum diseases.

### BACKGROUND OF THE INVENTION

Unfortunately, poor oral health affects millions of people every year. Poor oral health may result in symptoms ranging bad breath, tooth decay, and tooth discoloration, to more serious health problems, such as gum disease, tooth loss, heart disease, stroke, poorly controlled diabetes and preterm labor.

The presence of dental plaque, or simply plaque, in an oral cavity can lead to such oral and general health problems. Plaque can be defined as an organized, coherent, gel-like or mucoid material that includes microorganisms in an organic matrix derived from saliva and extracellular bacterial products such as glucans, fructans, enzymes, toxins, and acids. Plaque may also contain other cells, such as desquamated epithelial cells, and inorganic components, such as calcium and phosphate. In general, dental plaque is a bacterial accumulation. Generally transparent and sticky, plaque accumulates around the teeth at the cervical margin, and then grows apically.

Once plaque forms on a surface, the plaque resists removal, and usually can be removed only by mechanical means, such as, for example, by brushing and flossing the affected areas. If not removed, however, the presence of plaque can give rise to tartar formation, tooth decay, gingivitis, periodontitis, and other health problems.

Tartar is a hard, calcified plaque material that exhibits a yellowish or brownish color. Tartar forms as a result of minerals (e.g., those present in saliva and gum pockets) reacting with plaque material to form a rough calculus. Calculus generally arises from the nucleation of calcium phosphate, often in areas where the large salivary gland ducts secrete their saliva. As such, calculus can form on surfaces not covered by the oral mucosa (supragingival) or on surface located apical to the soft tissue margin of the gingiva (subgingival).

Tartar adheres to hard surfaces such as enamel, roots, and dental devices, such as dentures, bridges, crowns, and the like, and is generally more difficult to remove than plaque. Brushing and flossing are normally not sufficient to remove tartar from a surface.

If left untreated, tartar buildup can be problematic in several regards. For example, the rough, porous surface of tartar serves as a breeding ground for additional bacteria, which can calcify and form additional tartar. The bacteria growth can, in turn, lead to tooth decay, gum disease, tooth loss, as well as systemic health problems.

In addition to the health concerns, tartar is a cosmetic problem due to its discoloration of teeth. Namely, teeth can become yellowish or brownish color. Moreover, because the surface of tartar is rough and porous, the tartar absorbs colors from other sources (e.g., coffee, tea, tobacco, smoke, red wine and the like), and thus the presence of tartar exacerbates cosmetic tooth coloration typically associated with such other sources.

Typical methods of preventing tartar buildup include brushing with a tartar control toothpaste. Although such toothpastes, if used regularly, may prevent additional buildup of tartar, the toothpastes are not thought to be effective at removing existing tartar from tooth and device surfaces.

Methods of removing existing tartar typically include scaling or root planing, both of which are performed by dentists or hygienists with the aid of specialized tools. Although these techniques work well, they are relatively expensive and time consuming.

Furthermore, various methods for inhibiting tartar may cause damage to tooth enamel and/or to dental devices.

Accordingly, improved compositions and techniques for improving oral health are desired.

### SUMMARY OF THE INVENTION

In accordance with various aspects of the present invention, a composition for maintaining and promoting oral health is provided. The exemplary embodiments the invention provide a relatively inexpensive and safe composition for facilitating improved oral health and/or hygiene, such as through the reduction of tartar, plaque, gingivitis or gum diseases. In addition, composition is relatively easy to use or perform, does not require a visit to a dentist office, and does not damage the surface of enamel.

In accordance with various embodiments of the invention, a composition for protecting teeth, gums and other oral components is provided. In this regard, the composition assists in preventing and/or mitigating plaque build-up, tartar build-up and gingivitis, as well as reducing the effects of the same. Additionally, the composition provides a smoother, more comfortable “feel” to the teeth, gums and other oral components.

In accordance with additional embodiments of the invention, a composition includes one or more active ingredients to improve oral health and a viscous carrier designed to maintain the active ingredient(s) in contact with the surface for an extended period of time. Exemplary compositions have a viscosity greater than about 20,000 cp, preferably greater than 30,000 cp, and more preferably greater than about 35,000 cp. The viscosity of the compositions may range from about 20,000 to about 250,000 cp, preferably about 25,000 to about 100,000, and more preferably about 30,000 to about 50,000 cp. Exemplary active ingredients are configured for preventing the disposition of plaque, removing existing plaque, reducing tartar build-up and/or reducing an amount of tartar on a surface and include cetylpyridinium chloride (CPC), zinc salts, other antimicrobial agents, and other ingredients known to improve oral health.

In accordance with other embodiments of the invention, a composition includes a plurality of ingredients to improve oral health and a carrier that includes a thickening agent. The composition is configured to maintain the plurality of active ingredients in contact with a surface for an extended period of time. Exemplary active ingredients include CPC, zinc salts, other antimicrobial agents, and similar agents known to improve oral health. Exemplary thickeners include, for example, hydroxyethylcellulose, hydroxypropyl methylcellulose, carrageenan, guar gum, methylcellulose, methyethylecellulose, acceptable non-ionic thickeners, and/or the like.

In accordance with yet further embodiments of the present invention, a composition includes a colorant indicative healthy gingiva, an active ingredient for promoting oral health, and a carrier. In accordance with various aspects of this embodiment, the viscosity of the composition is greater than about 20,000, preferably greater than 30,000 cp, and more preferably greater than about 35,000 cp. The viscosity of the compositions may range from about 20,000 to about 250,000 cp, preferably about 25,000 to about 100,000, and more preferably about 30,000 to about 50,000 cp.

In accordance with yet other embodiments of the invention, an oral care composition includes one or more active ingredients, zinc, a thickener, and optional ingredients such as color and/or flavoring agents.

### DETAILED DESCRIPTION

The present invention provides an oral care composition that includes one or more active ingredients to maintain and/or promote oral health. The composition can be used to facilitate oral health for various animals, and is particularly well suited for the treatment of humans.

As used throughout this application, the term surface includes any surface on which tartar may form. Exemplary surfaces include teeth (both supragingival and subgingival) and dental devices such as bridges, crowns, fillings, braces, and the like.

In accordance with various embodiments, a composition includes an active ingredient and a viscous carrier. In this case, the composition is configured to maintain the active ingredient in contact with a surface for an extended period of time. As explained in greater detail below, the oral care composition is designed so that the composition remains in contact with the gums and teeth after application, but is low enough so that it can easily be applied to the crevices of the oral cavity using an applicator.

Exemplary active ingredients suitable for use with systems of the invention include one or more of the following: cetylpyridinium chloride (CPC), dicalcium phosphate dehydrate, hydrogen peroxide, sanguinaria extract, sodium bicarbonate, sodium lauryl sulfate, sodium fluoride, stannous fluoride, sodium monofluorophosphate (MFP), zinc salts such as zinc chloride, zinc acetate, zinc citrate, zinc oxide and zinc gluconate, alkyl dimethyl amine oxide, alkyl dimethyl glycine, eucalyptol, menthol, methyl salicylate, thymol, sodium citrate, peppermint oil, sage oil, polymethylsiloxane, polxamer, and stannous pyrophosphate. Other now known or hereafter devised actives may also be used. For example, any agent, which alone or in combination is able to prevent or alleviate the severity of problems associated with dentition may be utilized. Such may include anti-caries agents and the like; agents useful in reducing tooth hypersensitivity, such as potassium nitrate, strontium chloride and/or the like; and/or plaque and calculus reducing agents, such as, for example, chlorhexidine, quaternary ammonium compounds (e.g. benzethonium chloride, domphen bromide, etc.), triclosan, herbal compounds (e.g. sanguinarine), stannous salts, complex phosphates (e.g., pyrophosphates), SLS (e.g. sodium lauryl sulfate), hydrogen peroxide, and/or the like.

An amount of the active ingredient for use within compositions suitable for uses with the invention varies in accordance with the dosage size and particular ingredient(s). In general, each active or actives selected will be used in a suitably effective amount, generally on the order of less than about 10 wt %, and more preferably 5 wt % or less. An amount of active may also be desirably selected to be within government guidelines, such as guidelines by the Food and Drug Administration in the USA. In particularly preferred compositions, the active ingredient is present in an amount of about 0.001 wt % to about 1.5 wt %, within an amount of about 0.025 wt % to about 1.0 wt %, or even within an amount about 0.05 wt % to about 0.7 wt %. All percentages set forth herein are in weight percent of the total composition, unless otherwise indicated.

In accordance with one exemplary embodiment, the active ingredient comprises an antimicrobial agent such as CPC. CPC may be present in an amount of about 0.001% to about 1%, in an amount of about 0.01% to about 0.5%, or even in an amount of about 0.05% to about 0.25% or about 0.045% to about 0.1%. Alternatively, the CPC is present in an amount from about 0.001 to about 2%. In accordance with another exemplary embodiment, the active ingredient includes zinc gluconate. Zinc gluconate can be present in an amount of about 0.001% to about 1.5%, in an amount of about 0.01% to about 1.0%, or even in an amount of about 0.05% to about 0.75%.

In accordance with an exemplary embodiment, the composition includes a thickener to obtain the desired viscosity. Suitable thickening agents include substances which increases the viscosity of the composition, cause the composition to gel or coagulate, or the like, such as food-grade or pharmaceutical grade thickeners, including, for example, hydroxyethylcellulose, hydroxypropyl methylcellulose, carrageenan, sugar, guar gum, methylcellulose, methyethylecellulose, acceptable non-ionic thickeners, and the like. The thickener may be present in an amount of about 0.01 to about 10%, in an amount of about 0.1 to about 7%, or even in an amount of about 1% to about 5% or about 0.5% to about 3%.

The composition may also include a humectant such a glycerin, which may be present in an amount of about 0.01% to about 15%, preferably about 0.1% to about 10%, and more preferably about 1 to about 7%. When used, the humectant may facilitate maintaining the composition in a liquid form and may help maintain a desired viscosity. In addition, glycerin may facilitate maintaining one or more of the active ingredients in an ionic form and may facilitate the transport of the active ingredients through the composition.

The composition may also include a suitable diluent. Exemplary diluents include water, alcohols, and oils. For example, the composition can includes purified water in an amount of about 80% to about 99%, in an amount of about 85% to about 95%, or even in an amount of about 88% to about 92%.

The composition may also include sugar alcohols such as sorbitol and xylital, monnital that act as a sweetener and also as a humectant and/or emulsifier. When used, sorbitol or other sugar alcohol can be present in an amount of about 0.001% to about 0.5%, in an amount of about 0.01% to about 0. 1%, or even in an amount of about 0.025% to about 0.075%.

The composition may also include a natural or artificial sweetener such as sucralose, saccharin, ace-k, aspartame which, when included in the composition, can be present in an amount of about 0.001% to about 1.5%, in an amount of about 0.01% to about 1%, or even in an amount of about 0.25% to about 0.75%.

Colorants may also be added to the composition. For example, the composition can includes colorants, such that when the composition is applied to or proximate the gingiva, the composition has a color indicative of healthy gingiva-e.g., the composition can be pink in color. Such a composition having a color indicative of healthy gingiva can provide added incentive to users to continue using the composition, which in turn promotes improved health care and hygiene. Colorants may be present in any desired amount. For example, the colorants may include Red #33 and Red #40, available from Pylam in an amount of about 0.000005% to about 0.00018%, and more preferably about 0.00050% to about 0.00099% , respectively.

The composition may also include flavorants such as cinnamon oil, clove oil, mints, anise, citrus, fruits, and the like, which, when included in the formula are present in an amount of about 0.001% to about 2%, in an amount of about 0.01% to about 1%, or even in an amount about of about 0.1% to about 0.5%. By way of one particular example, a composition includes cinnamon bark oil present in an about of about 0.001% to about 2%, preferably about 0.01% to about 1%, and more preferably about 0.1% to about 0.5%; and clove bud oil in an amount of about 0.001% to about 2%, preferably about 0.002% to about 1%, and more preferably about 0.003% to about 0.0075%.

In accordance with exemplary embodiments, the composition is configured to maintain the composition in contact with a surface for an extended period of time, which has several advantages over traditional compositions. The composition preferably exhibits good microadhesion and/or the composition preferably is quite viscous. As such, in general, relatively small amounts of the composition and consequently the active agent(s) can be used to effectively provide oral health care or treatment. Additionally, the relatively high viscosity allows for relatively select placement of the composition on a surface. Exemplary compositions can comprise a viscosity greater than about 20,000 cp, greater than 30,000 cp, and at least greater than about 35,000 cp. The viscosity values as set forth herein are measured using a Brookfield, Model DV-II+ Pro viscometer, spindle #6, 10 RPM for 90 seconds at 25 C.

In accordance with another embodiment, the composition includes multiple active ingredients in a carrier, with and without a thickening agent. For example, the composition can include a plurality of any active ingredients and any thickening agents in the weight percents disclosed herein. The composition may also include any of the optional ingredients such as sweeteners, flavorants, and colorants as set forth herein. For example, in accordance with an exemplary embodiment, the composition includes CPC and zinc gluconate as the active ingredients, wherein the CPC is present in an amount of about 0.001% to about 1%, in an amount of about 0.01% to about 0.5%, or even in an amount of about 0.05% to about 0.25%; and wherein the zinc gluconate is present in an amount of about 0.001% to about 1.5%, in an amount of about 0.01% to about 1.0%, or even in an amount of about 0.05% to about 0.75%.

In accordance with other exemplary embodiments, the composition includes one or more active ingredients and a colorant indicative of healthy gingival, wherein the color of the composition is more than merely decorative to serve the function of encouraging those that use the product to continue to use the product because there is an immediate appearance, upon application of the composition, that healthy gingiva is achieved.

In accordance with another embodiment of the invention, a composition includes ionized zinc or a zinc salt in a viscous carrier. Exemplary zinc salts include zinc salts such as zinc gluconate, zinc acetate, zinc sulfate, zinc citrate, and zinc chloride. Zinc exhibits known health advantages as applied to viruses and colds. However, unexpected results have been obtained when zinc is applied to the teeth and gums. It is thought that contacting the teeth and/or gums with zinc in its ionic state enhances tissue regeneration, provides whitening effects, and reduces, and in some cases reverses, calculus formation on the teeth. Moreover, it is believed that zinc may synergistically enhance the advantages delivered by the active ingredients in solution. In accordance with an exemplary embodiment, the composition includes zinc gluconate, preferably, in an amount from about 0.001 to about 1.5 percent, more preferably from about 0.01 to about 1.0 percent, and optimally from about 0.05 to about 0.75 percent by weight.

In accordance with one aspect of this embodiment, the composition comprises an ingredient which maintains zinc in its ionic state. By maintaining zinc in this state during delivery of the oral care composition, it is believed that effective diffusion of the zinc and any active ingredients is facilitated into the membranes of the mouth tissue and gums, as well as the surfaces of the teeth. In accordance with an exemplary embodiment of this aspect of the invention, glycerin is provided, preferably, in an amount from about 0.1 percent to about 15 percent, more preferably from about 0.1 to about 10 percent, and optimally from about 1 to about 7 percent by weight of the oral care composition. The composition in accordance with this embodiment may also include any of the other ingredients noted herein in the amounts noted herein.

A method of facilitating oral health care includes providing a composition, accessing the composition, and selectively applying the composition to oral cavity surface.

The composition may be applied using a variety of methods such as with a swab, a syringe, or using any other means capable of dispensing the composition.

### SPECIFIC EXAMPLE

The following non-limiting example illustrates an exemplary composition in accordance with various embodiments of the invention. This example is merely illustrative, and it is not intended that the invention be limited to the example. Compositions in accordance with the present invention may include the ingredients listed below as well as additional and/or alternative inert materials, preservatives, and other constituents typically found in compositions for removing and/or preventing tartar buildup. In the case where exemplary inert materials and/or preservatives are listed, these ingredients are merely exemplary, and it is understood that other similar ingredients may be substituted for the materials listed in the examples below.

### EXAMPLE 1

### CPC and Zinc Gluconate

A pale light pink viscous gel, having a viscosity of about 40,000 cp, with cinnamon-clove characteristic odor and taste is formed by admixing the following ingredients in the amounts shown.

TABLE 1

| | | | Exemplary |
|---|---|---|---|
| Ingredient | Supplier | Weight % | Wt % Range |
| Purified Water | Copacker | 91.603 | 80-99 |
| Glycerin USP | Acme-Hardesty | 5.000 | 0.01-15 |
| HEC 250 HX | Hercules-Aqualon | 2.000 | 0.01-10 |
| Sorbitol | Roquette | 0.050 | 0.001-0.5 |
| Sucralose | Tate & Lyle | 0.400 | 0.001-1.5 |
| Cetylpyridinium | Dastech | 0.100 | 0.001-1 |
| Chloride | | | |
| Zinc Gluconate | American | 0.592 | 0.001-1.5 |
| USP | Ingredients/ | | |
| | Pharmachem Inc. | | |
| Cinnamon Bark | Berje | 0.250 | 0.001-2 |
| (Oil) | | | |
| Clove Bud (Oil) | Berje | 0.005 | 0.001-2 |
| Red #33 | Pylam | 0 | 0-0.00099 |
| Red #40 (1% sol.) | Pylam | 0.00099 | 0-0.00099 |

A clinical study comparing the composition of Example 1 to a placebo and a mouth rinse including CPC was conducted. Each group started with 25 people each. Prior to a three-week evaluation, one person from the Example 1 group and one person from the mouth rinse group dropped out. At the conclusion of a three-week test period, there was an observable difference in the quality and thickness of calculus in the group using the composition of Example 1, compared to the placebo and the mouth rinse. A significant disruption of calculus bridges or masses of calculus was observed in subjects using the composition of Example 1. A softening of the calculus was also observed for those using the composition of Example 1.

Finally, although exemplary embodiments of the present invention are set forth herein, it should be appreciated that the invention is not so limited. Various modifications, variations, and enhancements in composition and method set forth herein may be made without departing from the spirit and scope of the present invention.

We claim:

1. A composition to improve oral health, the composition comprising:
about 0 weight percent to about 1 weight percent of a first active agent comprising an antimicrobial agents;
about 0.001 weight percent to about 1.5 weight percent of a second active agent configured to improve oral health;
about 80 weight percent to about 99 weight percent diluent; and
about 0.01 weight percent to about 10 weight percent thickener.

2. The composition to improve oral health according to claim 1, wherein a viscosity of the composition is greater than about 20,000 centipoise.

3. The composition to improve oral health according to claim 1, wherein a viscosity of the composition is greater than about 30,000 centipoise.

4. The composition to improve oral health according to claim 1, wherein a viscosity of the composition is greater than about 35,000 centipoise.

5. The composition to improve oral health according to claim 1, further comprising a humectant.

6. The composition to improve oral health according to claim 1, further comprising a sugar alcohol.

7. The composition to improve oral health according to claim 1, further comprising a colorant indicative of healthy gingiva.

8. The composition to improve oral health according to claim 1, wherein the viscosity of the composition is about 25,000 cp to about 100,000 cp.

9. The composition to improve oral health according to claim 1, wherein the viscosity of the composition is about 30,000 cp to about 50,000 cp.

10. The composition to improve oral health according to claim 1, further comprising an essential oil.

11. The composition to improve oral health according to claim 10, wherein the essential oil comprises cinnamon bark oil.

12. The composition to improve oral health according to claim 10, wherein the essential oil comprises clove bud oil.

13. The composition to improve oral health according to claim 1, wherein the second active agent comprises a zinc salt.

14. The composition to improve oral health according to claim 1, wherein the composition comprises about 0.001 weight percent to about 1 weight percent cetylpyridinium chloride.

15. A composition for maintaining and improving oral health, the composition comprising:
about 0.001 weight percent to about 1.5 weight percent zinc salt; and
about 80 weight percent to about 99 weight percent diluent,
wherein the composition has a viscosity greater than about 20,000 cp.

16. The composition for maintaining and improving oral health of claim 15, further comprising about 0.001 weight percent to about 1 weight percent cetylpyridinium chloride.

17. The composition for maintaining and improving oral health of claim 15, wherein the viscosity is about 25,000 cp to about 100,000 cp.

18. The composition for maintaining and improving oral health of claim 15, wherein the viscosity is about 30,000 cp to about 50,000 cp.

19. A composition for maintaining and improving oral health, the composition consisting essentially of:
about 0.001 weight percent to about 1 weight percent of a first active agent;
about 0.001 weight percent to about 1.5 weight percent of a second active agent;
a diluent;
a thickener;
a humectant;
a sugar alcohol;
a sweetener;
a flavorant; and
a colorant.

20. The composition for maintaining and improving oral health of claim 19, wherein the flavorant comprises a plurality of essential oils.
